(19) United States
(12) Patent Application Publication
(10) Pub. No.: US 2015/0182392 A1
(43) Pub. Date: Jul. 2, 2015

(54) ADAPTABLE DIAPER SYSTEM

(71) Applicant: JoAnna Kay ANDERSON, Fort Wayne, IN (US)

(72) Inventor: JoAnna Kay ANDERSON, Fort Wayne, IN (US)

(21) Appl. No.: 14/588,835

(22) Filed: Jan. 2, 2015

Related U.S. Application Data
(60) Provisional application No. 61/923,099, filed on Jan. 2, 2014.

(51) Int. Cl. A61F 13/80; A61F 13/74; A61F 13/68

(52) U.S. Cl. CPC A61F 13/80; A61F 13/68; A61F 13/74

(57) ABSTRACT

A diaper system is provided for adapting an adjustable diaper to accommodate different individuals and their growth. The adjustable diaper may include an insertion assembly and a cover assembly. The cover assembly has an interior surface and an exterior surface on opposing sides of its wing shape, forming an anterior wing end and a posterior wing end interconnected by a crotch portion. Along and inward from each wing end of each surface the cover assembly may provide a plurality of waist fasteners of adjustably securing the adjustable diaper about the ever-growing waist of a wearer. The exterior surface between the anterior and the posterior wing ends provides a plurality of fasteners for adapting the height and width of the adjustable diaper. The insertion assembly may be removably secured within pockets formed on both ends of the interior surface of the cover assembly for easy replacement.

### CROSS-REFERENCE TO RELATED APPLICATION

This application claims the benefit of priority of U.S. provisional application No. 61/923,099, filed 2 Jan. 2014, the contents of which are herein incorporated by reference.

### BACKGROUND OF THE INVENTION

The present invention relates to the field of diapers and, more particularly, to a diaper system for adapting an adjustable diaper to accommodate different individuals and their growth.

Various types of diapers are currently produced and sold on the market. Most diapers tend to be disposable. When compared to reusable diapers, over the time an individual uses diaper, the disposables are exponentially expensive and wasteful as the discards fill our landfills. However, typically, reusable diapers are designed to fit individuals only in specific stage of growth.

As can be seen, there is a need for a diaper system for adapting an adjustable diaper to accommodate different individuals and all of their stages of growth. By adjusting the width of the diaper along with the waist and rise, we can ensure the best fit for all stages of a baby's growth.

### SUMMARY OF THE INVENTION

In one aspect of the present invention, an adjustable diaper cover assembly comprises: a sheet of material having an exterior surface and an opposing interior surface, wherein the sheet of material forms a posterior end and an anterior end interconnected by a crotch portion; a plurality of waist fasteners provided along and inward from the interior surface of the posterior end and along and inward from the exterior surface of the anterior end; a plurality of riser fasteners provided along the exterior surface of the crotch portion; and a plurality of width fasteners provided along the exterior surface between the posterior end and the anterior end.

In another aspect of the present invention, an adjustable diaper cover assembly comprises: a sheet of material having an exterior surface and an opposing interior surface, wherein the sheet of material forms a posterior end and an anterior end interconnected by a crotch portion, and wherein the crotch portion is tapered so that the sheet of material forms a generally winged shape; a plurality of waist fasteners provided along and inward from the interior surface of the posterior end and along and inward from the exterior surface of the anterior end; a plurality of cross-over waist fasteners provided along and inward from the exterior surface of the posterior end; a plurality of riser fasteners provided along the exterior surface of the crotch portion; a plurality of width fasteners provided along the exterior surface between the posterior end and the anterior end; and a pocket formed along both the posterior end and the anterior end of the interior surface, wherein each pocket has a shape complementary of the generally winged shape of the sheet of material.

These and other features, aspects and advantages of the present invention will become better understood with reference to the following drawings, description and claims.

### BRIEF DESCRIPTION OF THE DRAWINGS

FIG. 1 is a perspective view of an exemplary embodiment of an insertion assembly of the present invention;

FIG. 2 is a perspective view of an exemplary embodiment of the insertion assembly of the present invention;

FIG. 3 is a perspective view of an exemplary embodiment of the insertion assembly of the present invention;

FIG. 4 is a perspective view of an exemplary embodiment of the insertion assembly of the present invention;

FIG. 5 is a perspective view of an exemplary embodiment of a cover assembly of the present invention;

FIG. 6 is a perspective view of an exemplary embodiment of the cover assembly of the present invention;

FIG. 7 is an exploded view of an exemplary embodiment of the insertion assembly of the present invention;

FIG. 8 is a perspective view of an exemplary embodiment of the insertion assembly of the present invention, demonstrating use;

FIG. 9 is a perspective view of an exemplary embodiment of the insertion assembly of the present invention, demonstrating use;

FIG. 10 is a perspective view of an exemplary embodiment of the insertion assembly of the present invention, demonstrating use;

FIG. 11 is an exploded view of an exemplary embodiment of the present invention;

FIG. 12 is a perspective view of an exemplary embodiment of the present invention;

FIG. 13 is a perspective view of an exemplary embodiment of the present invention, demonstrating use;

FIG. 14 is a perspective view of an exemplary embodiment of the present invention, rotated 180 degrees relative to FIG. 13, demonstrating use;

FIG. 15 is a front view of an exemplary embodiment of the present invention;

FIG. 16 is a front view of an exemplary embodiment of the present invention, demonstrating use;

FIG. 17 is a front view of an exemplary embodiment of the present invention, demonstrating use;

FIG. 18 is a front view of an exemplary embodiment of the present invention, demonstrating use;

FIG. 19 is a front view of an exemplary embodiment of the present invention, demonstrating use;

FIG. 20 is a side view of an exemplary embodiment of the present invention, demonstrating use;

FIG. 21 is a side view of an exemplary embodiment of the present invention, demonstrating use;

FIG. 22 is a rear view of an exemplary embodiment of the present invention, demonstrating use; and

FIG. 23 is a rear view of an exemplary embodiment of the present invention, demonstrating use.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a diaper system for adapting an adjustable diaper to accommodate different individuals and their growth. The adjustable diaper may include an insertion assembly and a cover assembly. The cover assembly has an interior surface and an exterior surface on opposing sides of its wing shape, forming an anterior wing end and a posterior wing end interconnected by a crotch portion. Along and inward from each wing end of each surface the cover assembly may provide a plurality of waist fasteners of adjustably securing the adjustable diaper about the ever-growing waist of a wearer. The exterior surface between the anterior and the posterior wing ends provides a plurality of fasteners for adapting the height and width of the adjustable diaper. The insertion assembly may be removably secured within pockets formed on both ends of the interior surface of the cover assembly for easy replacement.

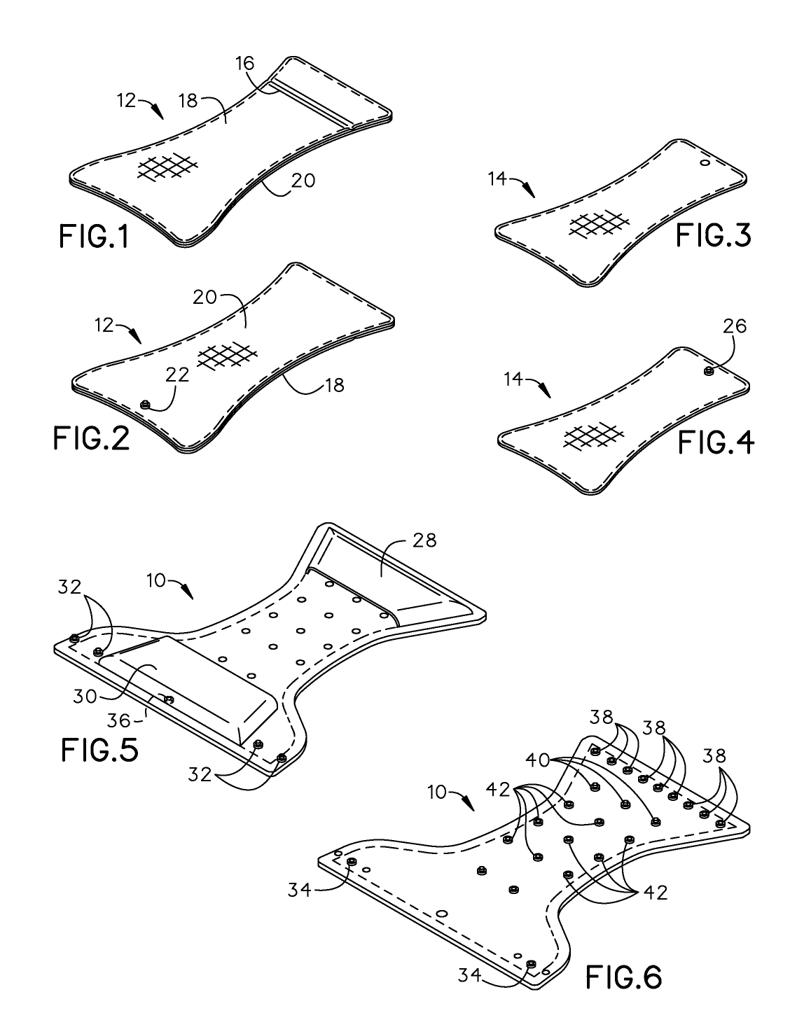

Referring to FIG. 1 through 23, the present invention includes a diaper system that embodies a method for adapting an adjustable diaper 50 to accommodate different individuals and their growth. The adjustable diaper 50 may include an insertion assembly 12 and a cover assembly 10.

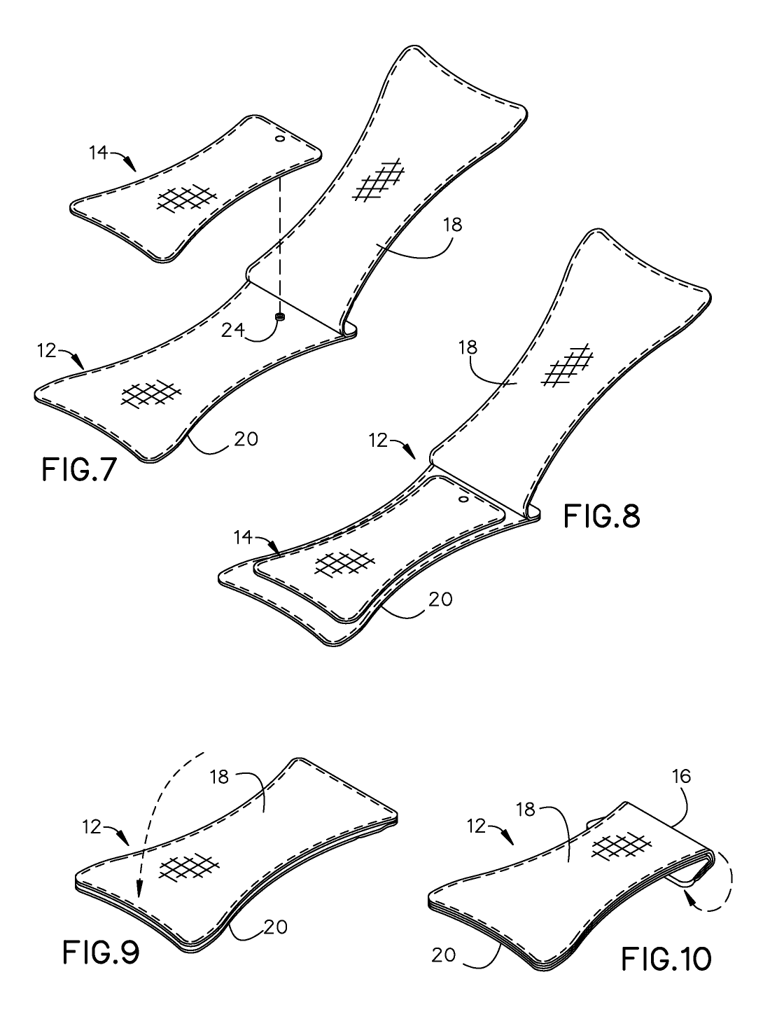

The insertion assembly 12 may be made of material that is suitably absorbent. The insertion assembly 12 may include at least one sheet of absorbent material formed into a contoured, general “wing” shape, as illustrated in FIGS. 1 through 4. Along and inward from one end, the insertion assembly 12 may provide a foldable portion adapted to fold about a fold line 16, as illustrated in FIGS. 1 and 10, so as to adjust the overall length of the insertion assembly 12 yet maintain its contoured, general wing shape. The insertion assembly 12 may include an individual side 18 and an opposing cover side 20. The fold line 16 is disposed on the individual side 18. Also on the individual side 18, near and inward from of the fold line 16, the insertion assembly 12 may provide a first flap fastener 24, as illustrated in FIG. 7. On the cover side 20, near and inward from the end opposite the fold line 16, the insertion assembly 12 may provide a second flap fastener 22, as illustrated in FIG. 2.

The insertion assembly 12 may include an infant sheet 14. The infant sheet 14 may have a substantially similar contoured, general “wing” shape of the other sheets of the insertion assembly 12, though smaller in size. The infant sheet 14 may provide a third flap fastener 26 adapted to detachably secure to the first flap fastener 24, as illustrated in FIG. 7. In an alternative embodiment, the third flap fastener 26 may detachably secure to a fourth flap fastener 36.

In certain embodiments, the insertion assembly 12 may provide two substantially similar sized sheets of absorbent material formed into a contoured, generally “wing” shape. The two sheets may be connected along cooperating ends so as to be pivotably separable, much like a notebook, wherein a first sheet provides the individual side 18 with folded line 16 and a second sheet provides the cover side 20 with the second flap fastener 22, and wherein the first flap fastener 24 is provided between the two sheets on the second sheet, as illustrated in FIG. 7. In some embodiments, the infant sheet 14 may be detachably secured between the first and second sheets to form a three-sheet insertion assembly 12, as illustrated in FIG. 8.

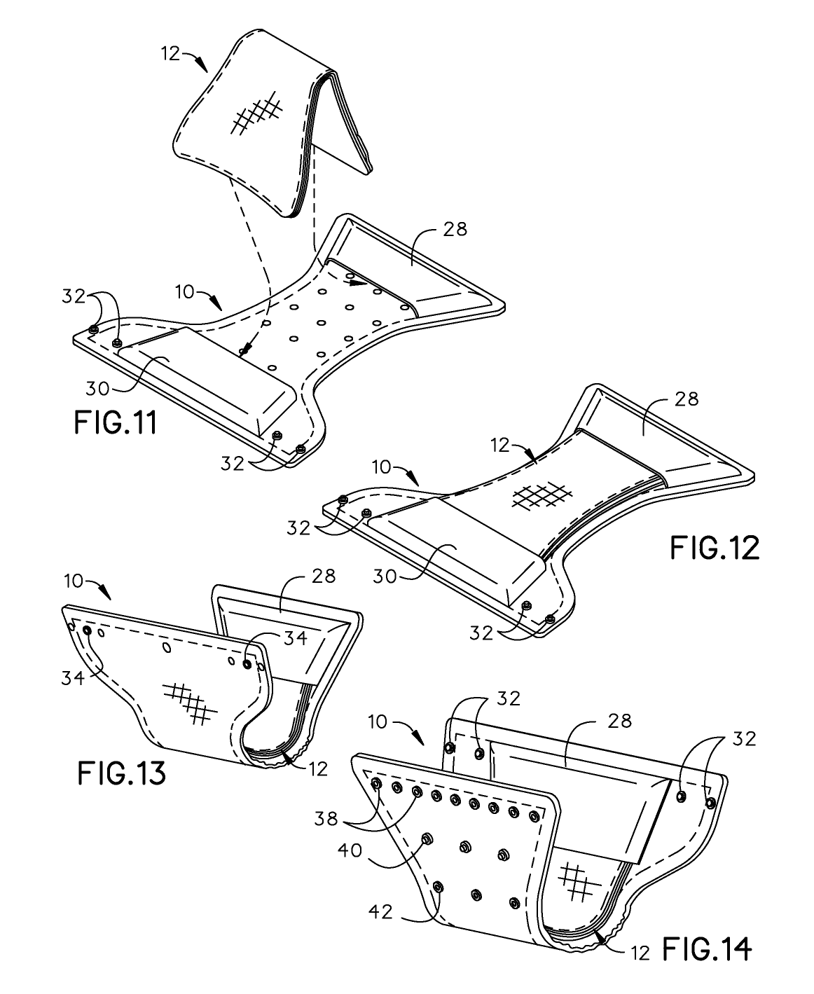

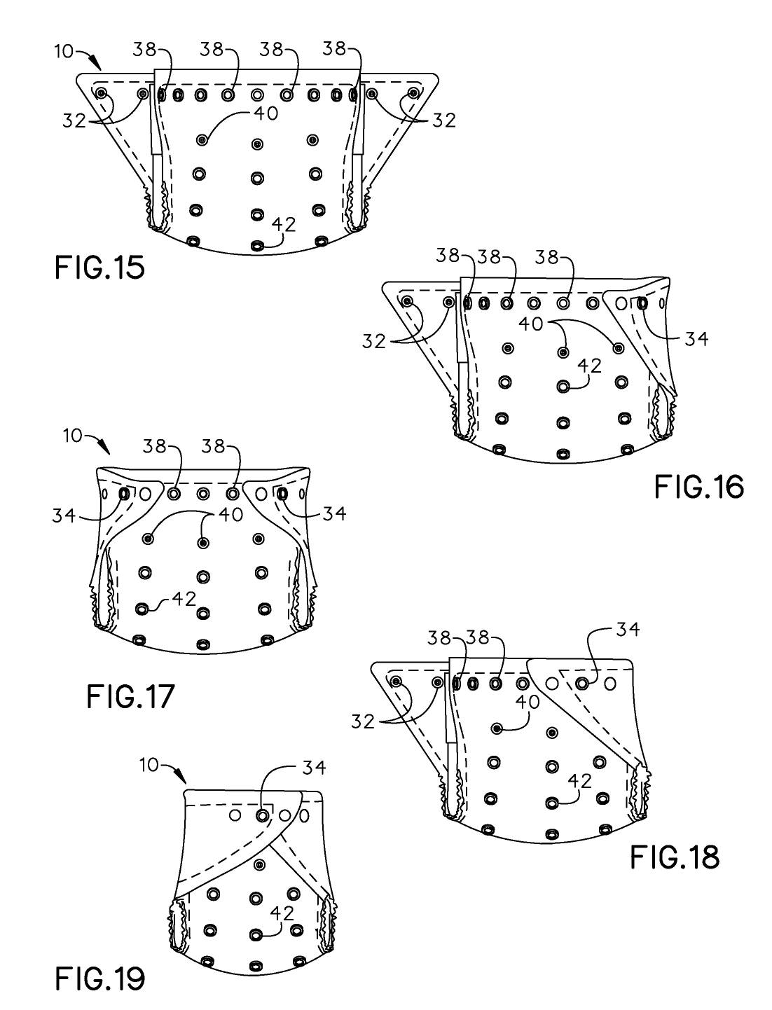

The cover assembly 10 may be made of material that is suitably water proof and/or moisture resistant. The cover assembly 10 may be formed into a contoured, generally “wing” shape so that the cover assembly 10 extends from a posterior (wing) end to and through a tapered crotch portion and to an anterior (wing) end. The posterior end may be dimensioned and adapted to have a greater “wingspan” than the anterior end so as to envelope the latter when in use, as illustrated in FIGS. 15 through 19. The cover assembly 10 provides an interior surface and an exterior surface. The interior surface may provide an anterior pocket 28 along the anterior end and an opposing posterior pocket 30 along the posterior end, as illustrated in FIG. 5. The pockets 28, 30 may be adapted for slidably receiving either end of the insertion assembly 12, as illustrated in FIG. 11. In certain embodiments, the interior surface may provide a fourth flap fastener 36 disposed within the posterior pocket 30, as illustrated in FIG. 5. The fourth flap fastener 36 may be adapted to removably connect to the second flap fastener 22 (or third flap fastener 26) so that the insertion assembly 10 may be detachably secured therein. The pockets 28, 30 may be dimensioned and adapted so that wing shape of the ends of the insertion assembly 12 are retained therein when the present invention is in a vertical orientation even in the absence of a connected fourth and second or third fastener 36, 22 or 26.

Along and inward from the posterior end of the interior surface, a plurality of first waist fasteners 32 may be disposed. Along and inward from the posterior end of the exterior surface, a plurality of second waist fasteners 34 (also known as cross-over fasteners 34) may be disposed. Along and inward from the anterior end of the exterior surface, a plurality of third waist fasteners 38 may be disposed. The first, second and third waist fasteners 32, 34, 38 may be adapted to detachably connect together, thereby forming a waist opening for adjustably securing the anterior end about the waist of a wearer, as illustrated in FIGS. 15 through 19. FIG. 19 illustrates how the first and second waist fasteners 32, 34 may be “cross-over” connected to form the smallest waist opening, whereby the third waist fasteners 38 may not be engaged.

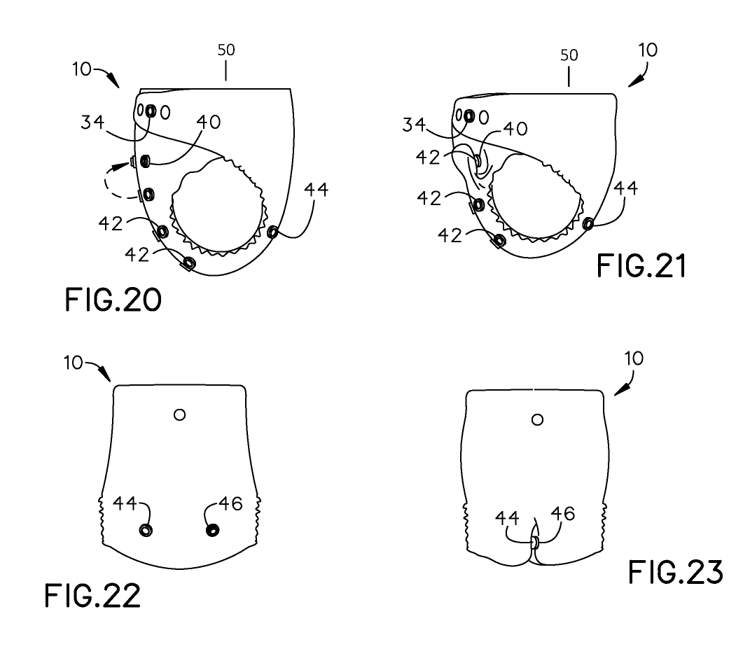

Inward from the plurality of third waist fasteners 38 a plurality of first riser fasteners 40 may be disposed, generally mirrored about a longitudinal axis of the cover assembly 10, as illustrated in FIG. 6. The crotch portion of the exterior surface may provide a plurality of second riser fasteners 42, as illustrated in FIG. 6. The first and second riser fasteners 40, 42 may be adapted to detachably connect together, thereby decreasing the rise/height of the cover assembly 10, as illustrated in FIGS. 20 and 21.

Along and generally mirrored about the longitudinal axis of the cover assembly 10, at least one first width fastener 44 and at least one second width fastener 46 may be provided between the posterior end and the anterior end. The first and second width fasteners 44, 46 may be adapted to detachably connect, thereby decreasing the width of the cover assembly 10.

A method of using the present invention may include the following. The cover assembly 10 and the insertion assembly 12 disclosed above may provided. Based on the size and dimensions of the desired adjustable diaper 50, a user may select the most suitably insertion assembly 12. For example, the infant sheet 14 may be used for a newborn wearer, or the “three-sheet” assembly may be used for toddlers, especially those producing excessive moisture. Similarly, the insertion assembly 12 may be folded about the fold line 16 to decrease the overall length of the insertion assembly 12. Then user may slide each end of the most suitable insertion assembly 12 into its cooperating pocket 28, 30 so that the cover side 20 of the insertion assembly 12 faces the interior surface of the cover assembly 10. The user may connect the fourth flap fastener 36 with the applicable fastener 26, 22 for further securing the insertion assembly 12.

Then the user may form a myriad of configurations of the adjustable diaper 50, having different height and width dimensions by engaging the appropriate arrangement of fasteners. For example, the user may wrap the posterior end of the cover assembly 10 about its anterior end so as to adjustable secure said ends about the waist of the wearer, fastening the appropriate waist fasteners 32, 34, 38 for the most secure and/or comfortable fit. Then the user may fasten the appropriate riser fasteners 40, 42 to adjust the height of the adjustable diaper relative to the wearer. Then the user may fasten the appropriate width fasteners 44, 46 to adjust the width of the adjustable diaper relative to the wearer. The adjustable diaper 50 may be at its “largest” height configuration when no width fasteners 44, 46 are engaged and/or at its “largest” width configuration when no riser fasteners 40, 42 are engaged.

Subsequent to use, the user may remove the insertion assembly 12 for discarding or washing and reuse. In certain embodiments, the insertion assembly 12 and the cover assembly 10 may be integrally formed.

It should be understood that the plurality of fasteners mentioned herein may be any fastener known in the art for fastening or removably securing one object to another including, for example, standard push-button snaps, Velcro-type fasteners, adhesive substances, combinations thereof, and the like. It should also be that the plurality of fasteners mentioned herein may be configured in any array and/or number, so long as the fasteners function in accordance with the present invention as described herein.

It should be understood, of course, that the foregoing relates to exemplary embodiments of the invention and that modifications may be made without departing from the spirit and scope of the invention as set forth in the following claims.

What is claimed is:

1. An adjustable diaper cover assembly comprising:
a sheet of material having an exterior surface and an opposing interior surface, wherein the sheet of material forms a posterior end and an anterior end interconnected by a crotch portion;
a plurality of waist fasteners provided along and inward from the interior surface of the posterior end and along and inward from the exterior surface of the anterior end;
a plurality of riser fasteners provided along the exterior surface of the crotch portion; and
a plurality of width fasteners provided along the exterior surface between the posterior end and the anterior end.

2. The adjustable diaper cover assembly of claim 1, further providing a plurality of cross-over waist fasteners provided along and inward from the exterior surface of the posterior end.

3. The adjustable diaper cover assembly of claim 1, wherein the crotch portion is tapered so that the sheet of material forms a generally winged shape.

4. The adjustable diaper cover assembly of claim 1, further providing a pocket formed along both the posterior end and the anterior end of the interior surface.

5. An adjustable diaper cover assembly comprising:
a sheet of material having an exterior surface and an opposing interior surface, wherein the sheet of material forms a posterior end and an anterior end interconnected by a crotch portion, and wherein the crotch portion is tapered so that the sheet of material forms a generally winged shape;
a plurality of waist fasteners provided along and inward from the interior surface of the posterior end and along and inward from the exterior surface of the anterior end;
a plurality of cross-over waist fasteners provided along and inward from the exterior surface of the posterior end;
a plurality of riser fasteners provided along the exterior surface of the crotch portion;
a plurality of width fasteners provided along the exterior surface between the posterior end and the anterior end; and
a pocket formed along both the posterior end and the anterior end of the interior surface, wherein each pocket has a shape complementary of the generally winged shape of the sheet of material.

6. An adaptable diaper system incorporating the adjustable diaper cover assembly of claim 5, comprising:
an insertion assembly comprising:
at least one sheet of absorbent material forming a shape complementary of the generally winged shape of the sheet of material so that the absorbent material has an absorbent posterior end and an absorbent anterior end interconnected by an absorbent crotch portion; and
a fold line near and inward from the absorbent anterior end.

7. The adaptable diaper system of claim 6, wherein the insertion assembly provides a second flap fastener along and inward from the absorbent posterior end, and wherein the adjustable diaper cover assembly provides a complementary fourth flap fastener within its posterior pocket.

8. The adaptable diaper system of claim 6, wherein the at least one sheet of absorbent material further comprises a first sheet and a second sheet of absorbent material pivotably joined along each absorbent posterior end.

9. The adaptable diaper system of claim 8, further providing a first flap fastener on the first sheet so as to face the second sheet.

10. The adaptable diaper system of claim 9, further providing an infant sheet having a third flap fastener that detachably connects to the first flap fastener.
